(19) United States
(12) Patent Application Publication
(10) Pub. No.: US 2002/0077577 A1
(43) Pub. Date: Jun. 20, 2002

(54) Typing protectant

(76) Inventor: Frederick L. Coger, Pittsburgh, PA (US)

Correspondence Address: JAMES RAY & ASSOCIATES, 2640 PITCAIRN ROAD, MONROEVILLE, PA, 15146, US

(21) Appl. No.: 09/943,817

(22) Filed: Aug. 31, 2001

Related U.S. Application Data
(63) Continuation-in-part of application No. 09/441,574, filed on Nov. 17, 1999, now abandoned.

(51) Int. Cl. A61F 13/00; A61F 13/06

(52) U.S. Cl. 602/64

(57) ABSTRACT

A device for protecting the wrist of typists using a keyboard of a computer terminal. The device includes a soft cushion removably attachable to the underside of a typist's wrist, and has opposed, resilient, curved extensions connected to said cushion means for receiving and engaging the wrist and for maintaining said cushion means in place between the wrist and a work surface of a workstation that includes the keyboard and computer terminal.

### CROSS REFERENCE TO RELATED APPLICATION

This application is a continuation-in-part of my co-pending application titled, “TYPING PROTECTANT”, filed on Nov. 17, 1999 and having Ser. No. 09/441,574, now abandoned.

### FIELD OF INVENTION

The present invention relates generally to devices for alleviation of the carpal tunnel syndrome that occurs in the wrists of typists who use computer terminals over extended periods of time, and more particularly to a soft device releasably attachable to the wrists of typists, the wrist device maintaining the wrists of the typist in an approximate in-line position with the hands of the typist.

### BACKGROUND OF THE INVENTION

It has become well known in industry and in the business world that typists who sit constantly at computer monitor keyboards and type for long periods of time develop what has come to be known as the carpal tunnel syndrome. It is a condition caused by compression of a nerve at the location where the nerve passes through the wrist (carpal) and into the hand, and is characterized especially by numbness, weakness, pain, tingling sensations in the middle and index fingers and thumb, and sometimes in all five fingers. The nerve, known as the median nerve, passes through a carpal (wrist) tunnel comprised of wrist bones and a transverse ligament. The median nerve is compressed in the carpal tunnel when the tunnel is constricted. When this occurs the above symptoms occur, as the carpal bones and ligament have substantially no “give”.

The level of a keyboard is somewhat higher than the plane of the work surface on which the keyboard rests so that the wrist bends when typing. Maintaining the wrist and hand in a generally straight line helps avoid the problem by avoiding contraction of the carpal tunnel caused by bending the wrist.

Elongated strips or pads of soft material have been developed for disposal in front of keyboards to limit bending of the wrists when typing. If the strip or pad is not firmly attached to the keyboard or work surface it can move about and cease to provide the needed wrist support. If the strip or pad is firmly fixed, it may not be located in a position suitable and comfortable for each individual typist. Typists have different size bands and length of fingers so that it is difficult to make and fixedly locate a strip of wrist supporting material in a manner suitable for all typists.

What is therefore needed is a support device that can be removably attached to the wrists of the typists such that the device is constantly present on the wrists and at a location appropriate for the individual typist to support the wrists in general alignment with the hands of typists.

### BRIEF SUMMARY OF THE INVENTION

It is therefore a primary objective of the invention to provide cushion means attachable to the wrists of typists that ensures straight line alignment of the wrists and hands at a location that is comfortable for the individual typist. This is accomplished by a soft device easily secured to each wrist of the typist for location between the lower surface of the wrist and the upper horizontal surface of a desk or table that provides a workstation that includes the keyboard of a computer terminal. The material of the device can be that of any soft material that retains its shape. One such material is a soft gel substance, such as silicone as used in breast implants, contained in a relatively soft container or jacket that has a shape suitable for supporting the wrist on a work surface of a workstation.

The wrist cushion of the invention can be provided with integral extensions or a strap that extend around and engage the wrist of the typist to maintain the cushion of soft material between the wrist and the surface of the workstation. Such extensions can have a resilient characteristic that inherently provides a size to fit substantially all wrists. A strap, preferably provided with a readily releasable adhesive such as VELCRO®, for ease of attachment and removal of the strap, can also be used to secure the cushion to the wrist of the typist.

### THE DRAWINGS

The invention, along with its objectives and advantages, will be best understood from consideration of the following detailed description in conjunction with the accompanying drawings in which:

FIG. 1 is a side elevation view of a person sitting at a workstation that includes a keyboard and computer terminal, the person wearing the wrist support device of the present invention;

FIG. 2 is a bottom plan view of a person's hand having the wrist device of the invention attached to the wrist of the person;

FIG. 3 is an end elevation view of the wrist device of the invention showing laterally disposed, integral extensions located to engage the wrist of a typist;

FIG. 4 is a transparent view of the right hand of a human, as viewed from the palm, showing the carpal tunnel and median nerve that divides in the hand and spreads out to the fingers and finger tips of the hand;

FIG. 5 is a plan view of a presently preferred embodiment of the invention; and

FIG. 6 is a cross sectional view taken along the lines VI-VI of FIG. 5.

### PRESENTLY PREFERRED EMBODIMENT OF THE INVENTION

Prior to proceeding to the following, more detailed description of the invention, it will be noted that identical components that have identical functions have been identified with the same reference numerals throughout the several views illustrated in the drawing figures for the sake of clarity and understanding of the invention.

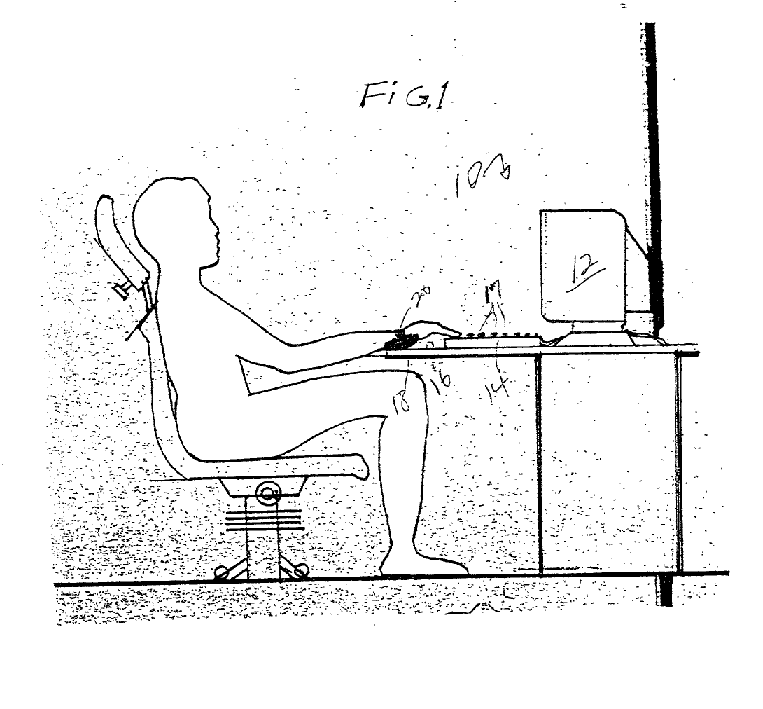

Referring now to the drawings, FIG. 1 thereof shows a typist sitting at a workstation 10 that includes a computer terminal and monitor 12 and a keyboard 14. The keyboard and terminal rest on a generally horizontal work surface 16 having a plane somewhat lower than the upper surface of the keyboard and keys 17 of the keyboard. As such, the wrists of the typist ordinarily rest on the lower work surface 16 and bend upwardly from the work surface to the keys of keyboard 14. This bending of the wrists is a primary cause of the carpal tunnel syndrome for typists that type constantly at computer workstations.

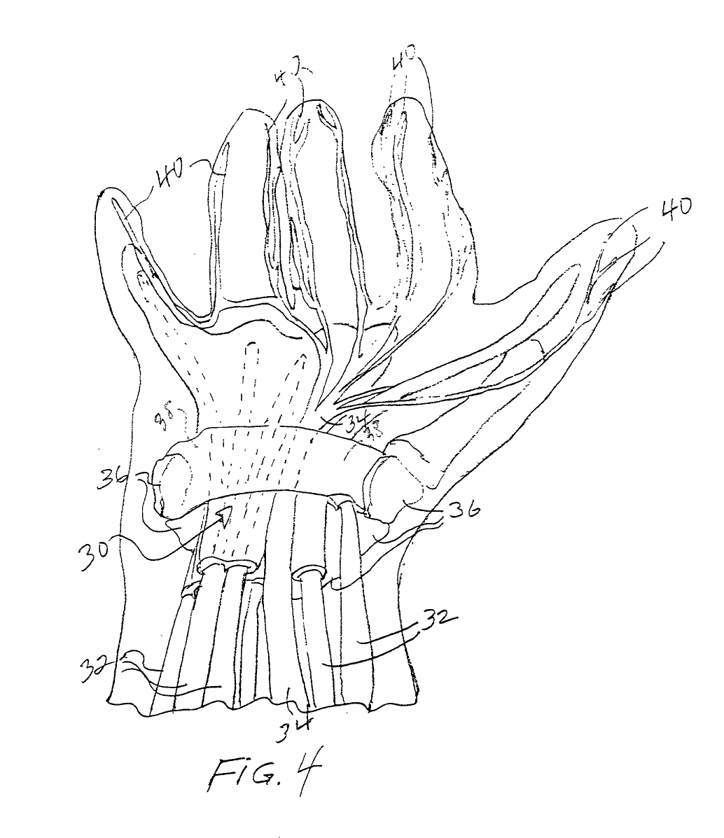

FIG. 4 of the drawings is a somewhat transparent view of the human hand that exposes the carpal (wrist) tunnel, generally designated by numeral 30, hand and wrist tendons 32, and the median nerve of the hand, as indicated by numeral 34. The view in FIG. 4 is that of the right hand, as seen from the palm side thereof.

The carpal tunnel 30 per se consists of carpal bones 36 that extend from the sides of tunnel 30 and beneath the tunnel in the view of FIG. 7. The carpal tunnel is completed (closed) by a transverse ligament 38 that extends from bones 36, located at opposed sides of the tunnel, to a position over the tendons 32 and median nerve 34.

Median nerve 34 divides and branches out to the four fingers and thumb of the hand, and terminates at the ends of the fingers and thumb as indicated by numeral 40. It is these nerve ends that receive the impact of fingers hitting against hard key surfaces in the typing process, and the median nerve 34 that receives the pressure when the carpal tunnel 30 becomes crowded and the wrist (carpal) is bent.

In the present invention, the typist in FIG. 1 is wearing the wrist protector of the invention in the form of a soft, resilient, cushion device 18 attached to his or her wrists (only one of which is visible in FIGS. 1 to 3) and located between the wrists and work surface 16 of workstation 10. Essentially, cushion devices 18 maintain the wrists of the typist in substantially straight line alignment with the hands of the typist. In this manner, the wrists of the typist are not bent upwardly when typing thereby alleviating a condition that causes the onset of the carpal tunnel problem.

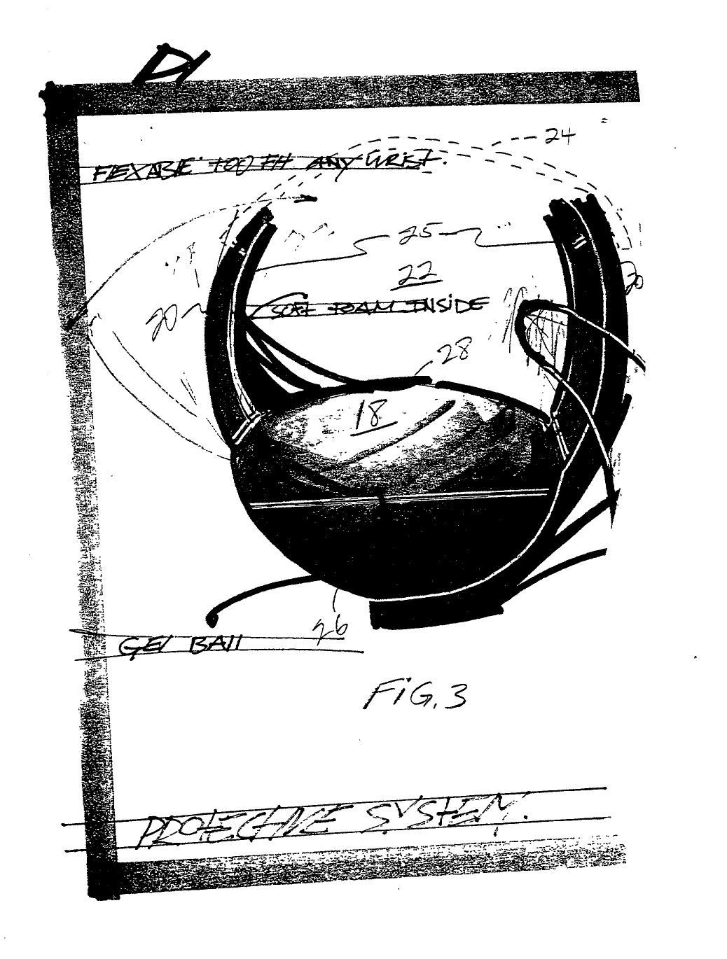

Cushion devices 18 are removably attachable to the wrists of typists by use of opposed curved extensions 20 of cushion 18. The extension form an open area and recess 22 adapted to receive a human wrist , and adapts to wrists of difference sizes because of a resilient, spring-like characteristic of extensions 20, i.e., larger wrists force the distal ends of extensions 20 outwardly while, at the same time, the recess smaller wrists, may not force the extensions outwardly in any substantial manner when the wrist occupies recess 22. A strap 24 of adhesive material such as VELCRO®, as shown in phantom in FIG. 3 of the drawings, can be used in conjunction with the distal ends of extensions 20 to secure cushion 18 to the wrist. Preferably, patches of adhesive material (not shown) can be located on strap 24 and on integral extensions 20 for securing the strap to the extension.

In addition, the inner curved surfaces of extension 20 are shown provided with a layer of soft material 25 in FIG. 3 to engage the wrists in a soft, compliant manner.

Cushion 18 can be a soft gel material, such as silicone, contained in a jacket or container made of a soft flexible but durable material. If the container is made of a suitable plastic material, each extension 20 can be integrally formed with the container in a molding and curing process. The soft gel can be disposed in the container and sealed therein in such molding process.

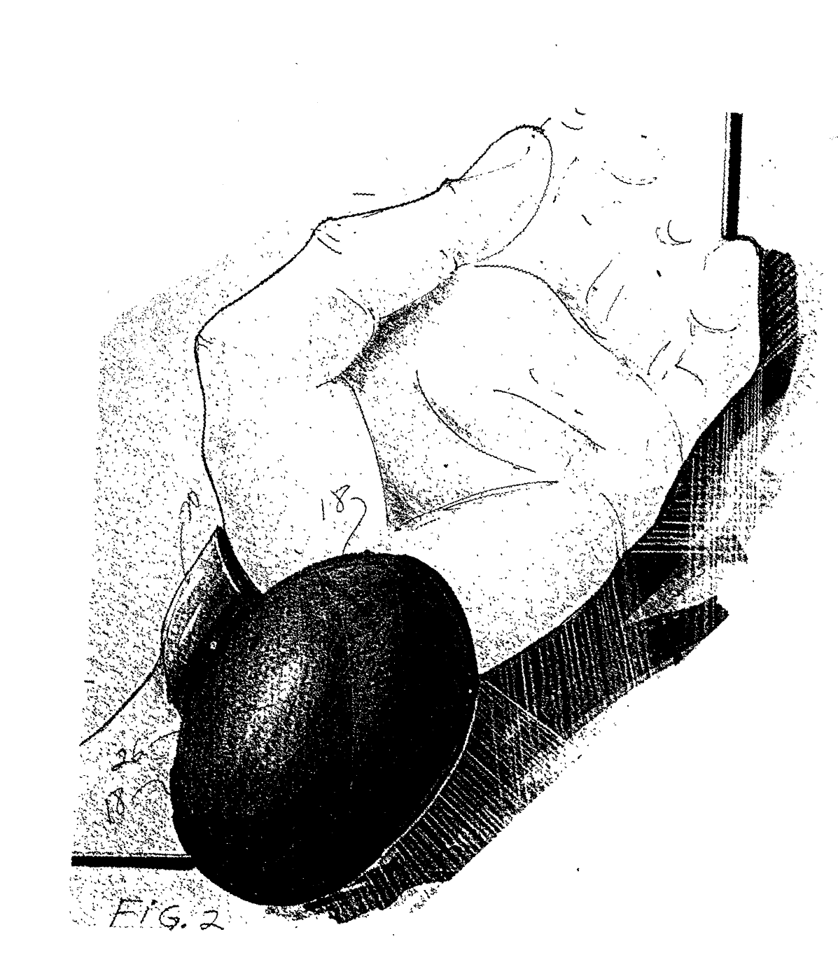

The shape of cushion 18 is preferably somewhat oblong in the longitudinal direction of the typists' wrists so that an appropriate area and length of resilient support is provided for the wrists. As depicted in FIGS. 1 and 2 of the drawings, cushion 18 can have an oval shape with a rounded lower surface 26 for resting on workstation surface 16 while an inner, wrist engaging surface 28 (FIG. 3) is somewhat more planar to provide a relatively flat resting surface for the wrists as cushion engages the underside of the wrist.

In viewing the hand depicted in FIG. 4 of the drawings, tendons 32 and median

nerve 34 pass directly through the carpal tunnel 30. The purpose of the present invention is to keep the wrist and hand in a straight line manner so that the media nerve is not crowded by undue bending of the hand relative to the wrist during typing, i.e., the tunnel 30 is maintained in a condition that provides maximum space with in the tunnel for accommodating the median nerve. In this manner, the above discussed carpal tunnel sensations ordinarily experienced by typists who type over long periods of time is not experienced.

In the process of resiliently supporting typists' wrists, cushion 18 also separates the wrists from the work surface 16 that is usually a hard surface. This, in addition, to the hand/wrist alignment function served by cushion 18, the cushion alleviates any impact of the wrists with such a hard surface during the typing process. The softer the environment for the hands and wrists of typists, the easier it is on typists' wrists in reference to the carpal tunnel syndrome. Cushion 18 and layers 25 of soft material are highly useful in this regard.

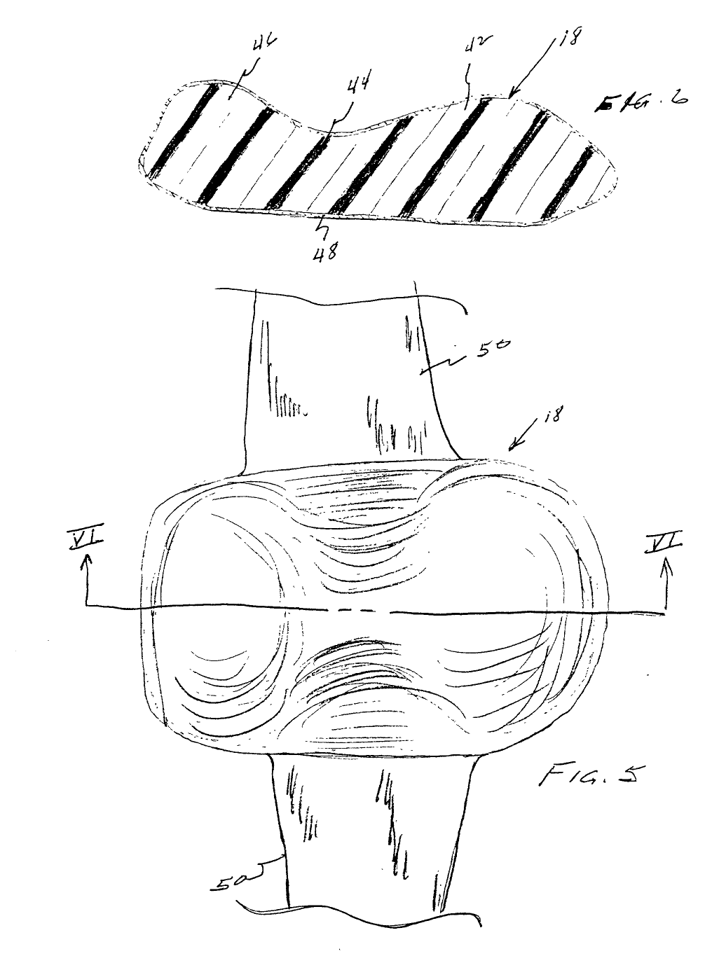

Now refer more particularly to FIGS. 5 and 6. Illustrated therein is a presently preferred embodiment of the invention. As can be seen the cushion 18 includes a first raised arcuate portion 42 disposed at a first end thereof which fits into the palm portion of the typist's hand. There is concave central portion 44 which receives the heel portion of the typist's hand and there is a second raised arcuate portion 46 which supports the wrists of such typist. As can also be seen, the cushion 18 is encased in a cover member 48 having straps 50 connected thereto to hold the cushion device 18 to the typist's hand and wrist.

While a presently preferred embodiment for carrying out the instant invention has been set forth in detail above, those persons skilled in the typing art to which this invention pertains will recognize various alternative ways of practicing the invention without departing from the spirit and scope of patent claims appended hereto.

What is claimed:

1. A device for protecting the wrist of typists using a keyboard of a computer terminal, comprising
a soft cushion means removably attachable to the underside of a typist's wrist, and
opposed, resilient, curved extensions connected to said cushion means for receiving and engaging the wrist and for maintaining said cushion means in place between the wrist and a work surface of a workstation that includes said keyboard and computer terminal.

2. The device of claim 1 wherein inside surfaces of the resilient extensions are provided with a layer of soft material for engaging said wrist.

3. The combination of claim 1 wherein the wrist device is elongated in the axial direction of the wrists and hands of typists.

4. The combination of claim 1 wherein the wrist device has an oval shape.

5. The combination of claim 1 wherein the wrist device has a rounded surface for resting on the work surface, and a substantially planar surface for disposal against the underside of a wrist of the typist.

6. The device of claim 1 wherein a flexible strap is removably attachable to said extensions.

7. The device of claim 6 wherein the flexible strap and resilient extensions are provided with adhesive material that allows the removable attachment of the strap and extensions.

8. A device for protecting the wrists of typists using a keyboard of a computer terminal, comprising:
a relatively soft cushion of material for location between a wrist of the typist and a work surface supporting the keyboard, and means for removably securing said soft cushion to a wrist of the typist.

9. A device for protecting the wrist of a person using one of a typewriter and a computer keyboard, said device comprising:
(a) a relatively soft flexible member having a first bulbous portion for supporting a palm of such person's hand using said device, a generally concave portion for supporting a heel of such hand of such person and a second bulbous portion for supporting a wrist of such person;
(b) a cover member encasing said relatively soft flexible member; and
(c) means secured to said cover member for securing said device to such person using said device.
